(19) United States
(12) Patent Application Publication
(10) Pub. No.: US 2007/0038125 A1
(43) Pub. Date: Feb. 15, 2007

(54) Oct-based imaging method

(75) Inventors: Martin Kleen, Furth (DE); Marcus Pfister, Bubenreuth (DE); Thomas Redel, Poxdorf (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/473,412

(22) Filed: Jun. 23, 2006

(30) Foreign Application Priority Data
Jun. 27, 2005 (DE) 10 2005 029 897.4

(51) Int. Cl. A61B 6/00

(52) U.S. Cl. 600/476

(57) ABSTRACT

The present invention relates to an imaging method with an OCT catheter for visualizing molecular functional processes in vulnerable plaques of a blood vessel of the blood vessel system of a patient, with OCT images of the contrast medium-marked vulnerable plaque being generated during continuously controlled movement of the light-emitting and light-absorbing OCT catheter head along the vulnerable plaque after the intravascular injection of a contrast medium into the blood vessel system and after the intravascular insertion of an imaging OCT catheter into the blood vessel comprising the vulnerable plaque.

### CROSS REFERENCE TO RELATED APPLICATIONS

This application claims priority of German application No. 10 2005 029 897.4 filed Jun. 27, 2005, which is incorporated by reference herein in its entirety.

### FIELD OF THE INVENTION

The present invention generally relates to catheter-based imaging, as used in medicine for the intravascular examination of patients. In this way, the present invention particularly relates to an OCT-based method, which enables and/or improves the visualization of functional molecular processes.

### BACKGROUND OF THE INVENTION

Optical Coherence Tomography (OCT) is an imaging method for displaying up to 2 mm thick tissue structures with a resolution of approximately 15 μm. Similar to ultrasound diagnostics, in which acoustic structure transitions are reconstructed into images, in OCT optical structure transitions are displayed in two-dimensional cross-sectional depth images. The transitions are characterized by the tissue-specific refraction indices in each instance. Tissue analysis with OCT is possible in a non-contact manner, which enables high resolution intravascular examinations of the vascular system of the heart, the oesophagous, the trachea etc. for instance particularly with the integration of this technology in catheters and endoscopes. However, the blood-filled vessels to be examined must first be rinsed out in order for imaging to be possible, since blood is not transparent with the light wavelength used.

For intravascular OCT imaging, the OCT catheter inserted into the vessel and/or hollow organ to be examined is slowly removed in the form of a continuously controlled movement, while the laser light scattered and/or reflected at the vessel inner wall is injected back into the optical waveguide of the catheter, fed to an evaluation unit and analyzed and processed in an imaging fashion. A “stack” of two-dimensional cross-sectional images is hereby achieved, which can in principle be combined to form a three dimensional data set offline, in other words after the actual measurement. The current two-dimensional cross-sectional image, which generally features a very high spatial resolution, is however usually visible on the screen. Depending on the distance from the catheter, in the x, y direction (transverse plane in relation to the catheter axis) this is up to 40 μm and in the z-direction (catheter axis) depending on the frame rate (as a function of the rotation speed of the laser beam coming from the catheter tip) and catheter removal speed it is likewise 40 to 100 μm. The quality of the images is heavily dependent inter alia on the degree to which the rinsed vessel is free of blood residues at the time of the recording. The images (similar to those of the ultrasound imaging) are generally very heavily distorted and possibly even subject to artifacts, due to blood residues but also to the method itself, which is why it is currently difficult for a doctor looking for the most minimal pathological changes to make a reliable diagnosis.

WO 02/067767 A2 discloses different contrast media for visualizing molecular functional processes in vulnerable plaques, with OCT also being cited as a possible imaging method.

US 2004/0258759 likewise discloses the use of contrast media, which improve imaging in Optical Coherence Tomography methods, in particular for visualizing vulnerable plaques.

DE 103 2 3217 A1 likewise deals with an OCT catheter and likewise mentions its use with vulnerable plaques.

### SUMMARY OF THE INVENTION

The object of the present invention is to provide a method which enables catheter-based OCT imaging in respect of morphological contrast as well as functional molecular processes to be further improved.

This object is achieved according to the invention by the features of the independent claim. The dependent claims form the central concept of the invention in a particularly advantageous manner.

In accordance with the invention, an OCT catheter-based imaging method is thus claimed for the purpose of visualizing molecular functional processes in vulnerable plaques of a blood vessel of the blood vessel system of a patient, with OCT images of a contrast medium-marked vulnerable plaque being generated with continuously controlled movement of the light-emitting and light-absorbing OCT catheter head along the vulnerable plaque after the intravascular injection of a contrast medium into the blood vessel system and after the intravascular insertion of an imaging OCT catheter into the blood vessel comprising the vulnerable plaque, with location of the vulnerable plaque and thus the position of insertion being effected by means of a preceding non-invasive magnetic resonance tomography method using the same contrast medium.

The contrast medium thereby advantageously comprises paramagnetic iron oxide particles.

In accordance with the invention, quantification of the macrophages is further carried out by comparing the contrast medium concentration in healthy vessel segments with the contrast medium concentration in the vulnerable plaque.

### BRIEF DESCRIPTION OF THE DRAWINGS

Further advantages, features and characteristics of the present invention are described below based on exemplary embodiments with reference to the accompanying drawings, in which;

FIG. 1 shows a schematic representation of the principle of the optical coherence tomography,

FIG. 2 shows a schematic representation of the technical arrangement of a catheter-based OCT method,

FIG. 3 shows a two-dimensional OCT image of a healthy blood vessel, and

FIG. 4 shows a schematic representation of a coronary sclerosis in the early stages (left), as well as an advanced coronary sclerosis with vulnerable plaques (right).

### DETAILED DESCRIPTION OF THE INVENTION

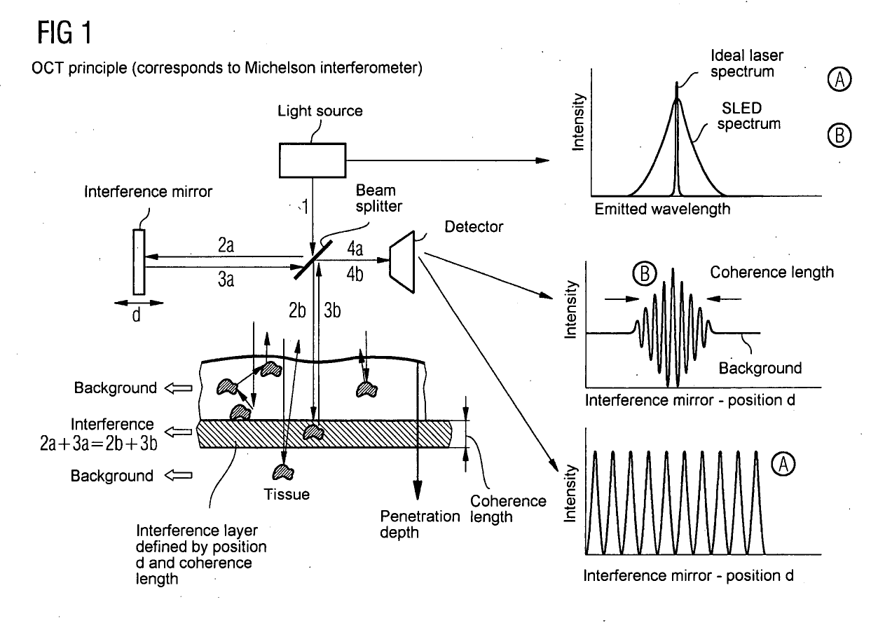

The principle of optical coherence tomography is to be explained below with reference to FIG. 1. The principle corresponds to the mode of operation of a Michelson interferometer. A light beam (e.g. laser beam) 1 emitted from a more or less coherent light source is divided by a beam splitter in the form of a semi-transparent mirror into two sub-beams 2a, 2b. The sub-beam 2a is directed onto an interference mirror, such that it strikes the beam splitter again in the form of a reflected beam 3a, penetrates said beam splitter and strikes a detector as beam 4a. In contrast the sub-beam 2b transmits the beam splitter immediately and is directed onto the tissue to be examined which comprises reflection and scatter centers, at which it strikes the beam splitter again in the form of a reflected beam 3b. This time however it is reflected by this and strikes the detector similarly as beam 4b. With the interference condition 2a+3a=2b+3b, the beam 4b coming from the interference layer interferes with the beam 4a as shown in an interference pattern in the detector image.

The (penetration) depth of the interference layer is defined by the position d of the interference mirror in relation to the beam splitter, which can preferably be periodically varied for the purpose of scanning in layers. The thickness of the interference layer and thus the (tissue structure) resolution of OCT imaging is thus dependent on the spectrum of the light source used due to the coherence length of the light used. If an “ideal” laser is used for instance (A), which emits coherent light in the form of “infinitely long wave trains” of a single spectral line (spectrum A), the coherence length would be infinite and an interference signal would result in the detector according to sub-image A. If a light source with a certain spectral width is used, for instance an SLED (Super Luminescent Light Emitting Diode (Spectrum B)), the interference pattern in the detector is restricted to a region corresponding to the coherence length in accordance with sub-image B. Light which is reflected or scattered at tissue structures, which are not located in the region of the interference layer, but is still injected via the beam splitter, does not fulfill the interference condition and thus does not contribute to any interference. It is only to be recognized as a consistent background, onto which the actual interference signal is modulated.

Technically speaking, a tissue region to be examined can thus be scanned by translating and/or rotating the sub-beams 2b/3b whilst at the same changing the position of the interference mirror. In this way, a depth scan (variation of d) is referred to as a so-called A-scan as in ultrasound technology.

To achieve a two-dimensional image, the tissue is scanned laterally. The amplitude values of the individual A-scan are displayed in logarithmized grey scales or pseudocolor values. The resulting image is then referred to as a B-scan. A one second measurement time is required for a B-scan comprising several hundred individual A-scans.

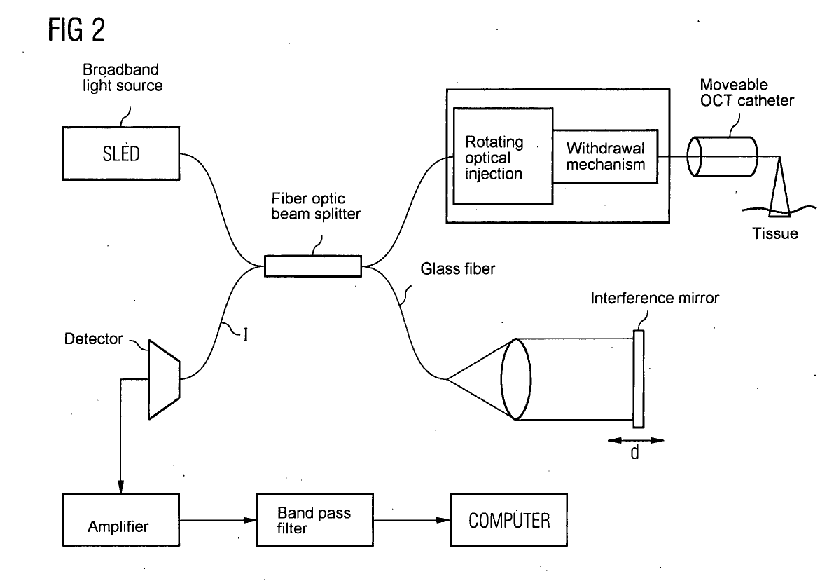

FIG. 2 shows a schematic representation of the technical arrangement for implementing catheter-based OCT imaging. An SLED serves as a broadband light source, the coherent light of which is fed to a fiber-optic beam splitter via glass fibers. On the one hand this effects a coherence-producing division of the light into one beam, which is projected onto the interference mirror, and one beam, which is injected into a rotating glass fiber along the axial OCT catheter axis via a rotating injection mirror and is extracted at the catheter tip via a mirror arranged perpendicular to the axial catheter axis and is projected vertically onto the tissue. On the other hand, it guides the light reflected by the interference mirror and by the tissue, which is partially interfering in region I, to the detector, the measurement signal of which is amplified, filtered and processed by means of a computer so that it can be displayed suitably on a screen.

The aim of catheter-based imaging (and/or endoscopy) is to display the inside of hollow organs, in particular of blood vessels, with as little damage as possible. With catheter-based OCT imaging, this is achieved in that the light emitted from the glass fiber at the catheter tip is extracted radially (at an angle of 90°) in respect of the axial axis of the catheter and is thus projected vertically onto the inner wall of the vessel with an axial catheter guide.

By rotating the axial catheter glass fiber, a cylindrical (and/or an annular) segment is scanned. The depth scan is carried out by moving the interference mirror considerably faster than the rotation speed of the fiber (approx 0.5- 20 MHz). To scan a longer tissue segment of interest, the OCT catheter is withdrawn in a regular and slow manner (approximately 0.5-2 mm/s) using an optionally automatic withdrawal mechanism, moving the catheter tip axially, with simultaneous rotation of the fiber and simultaneous variation of the interference mirror (similar to the table movement in spiral CT imaging).

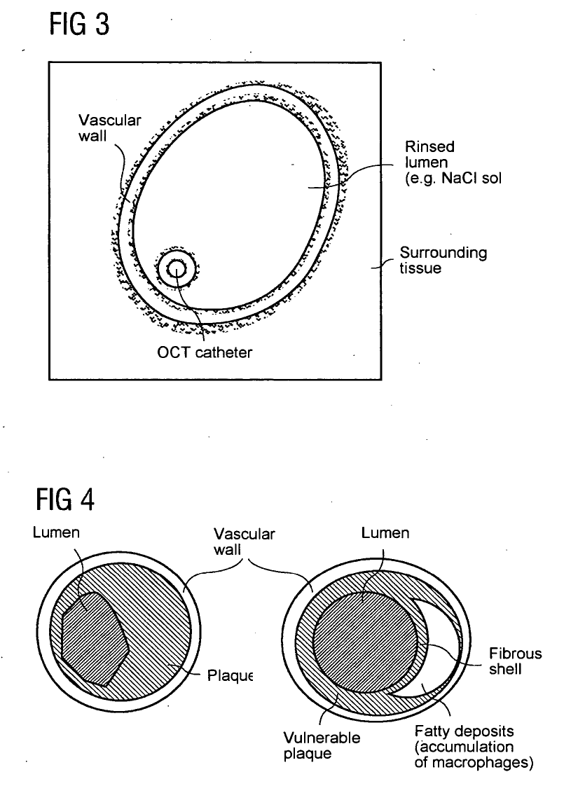

An examination of a healthy coronary artery results for example in an image series of cross-sectional images according to FIG. 3. The lumen (shaded vessel interior) is visible, with the OCT catheter located to the bottom right (self imaging). The vessel wall is shown in light shades, clearly delineating the blood vessel from the surrounding tissue. The brightness measure is defined by changing the optical refraction index. Light structures thus represent more reflective regions and/or regions with a distinctive non-continuous transition of the refraction index.

The fact that the entire vessel wall is visible in FIG. 3 is thus due to the blood being removed and being replaced by a medium (saline solution for instance) that is transparent at the OCT light wave length (λ≈1300 ±20 nm; short wave ultra red) used prior to image acquisition. Blood is removed during the OCT imaging of blood vessels in that an occlusion balloon attached upstream of the catheter is inflated after insertion of the OCT catheter, to stop the blood flow before the site to be examined. A sterile saline solution is injected distally in respect of the balloon via the catheter, thereby cleaning the vessel of blood, in other words rinsing it. In a further embodiment (e.g. with more complex vascular anatomies), two or more occlusion balloons can be used.

Catheter-based OCT imaging is particularly revealing in the case of atherosclerotic narrowing of blood vessels, which results in a fatal cardiac infarct in the event of a vessel rupture (unstable angina pectoris, acute myocardial infarct). Such narrowing initially arises as a result of deposits on and/or in the vascular wall of the blood vessel and is generally referred to as “plaque”. The lumen is significantly reduced by such a plaque (FIG. 4, left image), which interferes with the blood flow and thus the blood supply to the connected organs. In an advanced and much more dangerous stage, fatty deposits (lipid pool) form in such a plaque with a thin fibrous shell between the lumen and lipid pool, generally resulting in inflammation and thus causing the accumulation of macrophages (engulfing cells). A plaque of this type which tends to cause rupture or erosion (thrombosis) (FIG. 4, right image), is referred to as “vulnerable plaque” or also as “unstable atherosclerosis” and/or as “late stage atherosclerosis”.

Catheter-based OCT allows not only the vessel lumen but also the vessel wall to be imaged and a stage classification of the atherosclerosis (FIG. 4) to be carried out.

The present invention thus combines OCT with the use of specific contrast media, so as generally to increase the morphological contrast on the one hand and to make molecular functional processes visible on the other hand.

In accordance with the invention the contrast medium for instance comprises small paramagnetic iron oxide particles (Super Paramagnetic Iron Oxide—SPIO) with diameters on average in the range of 150 to 250 nm. In principle however, each specific molecule of this order of magnitude can be used as contrast medium, provided it accumulates in the structure to be examined such that a higher concentration is present here compared with its surroundings and provided it has a different optical refraction index from its surroundings.

Macrophages in particular have the characteristic of preferably absorbing particles of this type (in particular SPIO particles, as a result of which they and/or their activity is visible in OCT and possibly also in other imaging methods (e.g. MRT). From this increased absorption, it is clear that the macrophages have metabolism, in other words are active, and ultimately effect a breakdown of the fibrous shell, which eventually results in the cardiac infarct.

Since a relationship exists between the number of macrophages and the absorbed particles, a quantification can be carried out by comparing particle concentrations (SPIO concentrations) in healthy vessel segments (e.g. on the way to the examination area) and the particle concentration in the pathogenic area to be examined (e.g. in the vulnerable plaque), in other words a conclusion can be drawn about the macrophages there and thus the stage of the disease. The contrast medium-based visualization of the macrophages thus significantly simplifies the diagnostic evaluation of vulnerable plaque by means of OCT.

Since the examination with a non-invasive method (e.g. MRT or US) always precedes an invasive OCT examination, it would be advantageous in terms of supplementing both examination methods to use a contrast medium which can be used equally for both methods.

Since the majority of these contrast media are only broken down very slowly and thus remain in the body for a long time, may be expedient to administer the contrast medium for an MRT examination preceding the OCT examination, for instance in order to locate atherosclerosis vessel segments initially in a non-invasive manner.

1-3. (canceled)

4. An imaging method using an Optical Coherence Tomography catheter for visualizing a molecular functional process in a vulnerable plaque of a blood vessel in a blood vessel system of a patient, comprising:
injecting a contrast medium intravascular into the blood vessel system;
inserting the Optical Coherence Tomography catheter intravascular into the blood vessel comprising the vulnerable plaque, a location of the vulnerable plaque identified by a preceding non-invasive magnetic resonance tomography method using the same contrast medium; and
generating an image of the contrast medium marked vulnerable plaque by continuously controlling a movement of a light-emitting and light-absorbing Optical Coherence Tomography catheter head along the vulnerable plaque.

5. The method as claimed in claim 4, wherein the contrast medium comprises a paramagnetic iron oxide particle.

6. The method as claimed in claim 5, wherein a diameter of the paramagnetic iron oxide particle is in a range of 150 nm to 250 nm.

7. The method as claimed in claim 4, wherein the contrast medium comprises a molecule:
with a diameter between 150 nm to 250 nm,
accumulating a higher concentration in the vulnerable plaque compared with a surrounding area of the vulnerable plaque, and
having an optical refraction index that is different from the surrounding area of the vulnerable plaque.

8. The method as claimed in claim 4, wherein a quantification of a macrophage is carried out by comparing a concentration of the contrast medium in a healthy vessel segment with a concentration of the contrast medium in the vulnerable plaque.

9. A device for visualizing a molecular functional process in a vulnerable plaque of a blood vessel in a blood vessel system of a patient, comprising:
an injector for intravascular injecting a contrast medium into the blood vessel system;
an Optical Coherence Tomography catheter for intravascular inserting the Optical Coherence Tomography catheter into the blood vessel comprising the vulnerable plaque, a location of the vulnerable plaque identified by a preceding non-invasive magnetic resonance tomography method using the same contrast medium; and
a light-emitting and light-absorbing Optical Coherence Tomography catheter head for generating an image of the contrast medium marked vulnerable plaque by continuously controlling a movement of the Optical Coherence Tomography catheter head along the vulnerable plaque.

10. The device as claimed in claim 9, wherein the contrast medium comprises a paramagnetic iron oxide particle.

11. The device as claimed in claim 10, wherein a diameter of the paramagnetic iron oxide particle is in a range of 150 nm to 250 nm.

12. A contrast medium used in an Optical Coherence Tomography image method for visualizing a molecular functional process in a vulnerable plaque of a blood vessel in a blood vessel system of a patient, comprising:
a molecule having a diameter between 150 nm to 250 nm,
wherein the molecule accumulates a higher concentration in the vulnerable plaque compared with a surrounding area of the vulnerable plaque and has an optical refraction index that is different from the surrounding area of the vulnerable plaque.

13. The contrast medium as claimed in claim 12, wherein the contrast medium is a paramagnetic iron oxide particle.
